(19) United States
(12) Patent Application Publication
(10) Pub. No.: US 2023/0033442 A1
(43) Pub. Date: Feb. 2, 2023

(54) SYSTEMS AND METHODS OF USING SELF-ATTENTION DEEP LEARNING FOR IMAGE ENHANCEMENT

(71) Applicant: Subtle Medical, Inc., Menlo Park, CA (US)

(72) Inventors: Lei XIANG, Menlo Park, CA (US); Enhao GONG, Menlo Park, CA (US); Tao ZHANG, Menlo Park, CA (US); Long WANG, Menlo Park, CA (US)

(21) Appl. No.: 17/706,163

(22) Filed: Mar. 28, 2022

Related U.S. Application Data
(63) Continuation of application No. PCT/US2020/053078, filed on Sep. 28, 2020.
(60) Provisional application No. 62/908,814, filed on Oct. 1, 2019.

(51) Int. Cl. G06T 7/00; G06T 3/40; G06V 10/771; G06V 10/82

(52) U.S. Cl. CPC G06T 7/0012; G06T 3/4053; G06V 10/771; G06V 10/82; G06T 2207/10088; G06T 2207/10104; G06T 2207/20081; G06T 2207/30168

(57) ABSTRACT

A computer-implemented method is provided for improving image quality. The method comprises: acquiring, using a medical imaging apparatus, a medical image of a subject, wherein the medical image is acquired with shortened scanning time or reduced amount of tracer dose; applying a deep learning network model to the medical image to generate one or more feature attention maps a medical image of the subject with improved image quality for analysis by a physician.

### CROSS-REFERENCE TO RELATED APPLICATION

This application is a continuation of International Application No. PCT/US2020/053078 filed on Sep. 28, 2020, which claims priority to U.S. Provisional Application No. 62/908,814 filed on Oct. 1, 2019, the content of which is incorporated herein in its entirety.

### BACKGROUND

Medical imaging plays vital role in health care. Various imaging modalities such as Positron Emission Tomography (PET), Magnetic Resonance Imaging (MRI), ultrasound imaging, X-ray imaging, Computed Tomography (CT) or a combination of these modalities aid in prevention, early detection, early diagnosis and treatment of diseases and syndromes. Image quality may be degraded, and the images may be contaminated with noise due to various factors such as physical limitation of the electronic devices, dynamic range limit, noise from the environment and the movement artifacts due to movement of patient during imaging.

There is an ongoing effort to improve the quality of images and reduce various types of noise such as aliasing noise and various artifacts such as metal artifacts. For example, PET has been widely applied in clinics for diagnosis of challenging diseases, such as cancer, cardiovascular disease, and neurological disorders. Radiotracers are injected into patients prior to PET exams, introducing inevitable radiation risks. To tackle the radiation problem, one solution is to reduce the tracer dose by using a fraction of full dosage during the PET scans. Since PET imaging is a quantum accumulation process, lowering the tracer dose inevitably involves unnecessary noises and artifacts, thus degrading the PET image quality to a certain extent. As another example, compared with other modalities (e.g., X-ray, CT or ultrasound) conventional PET may take longer time, sometimes tens of minutes, for data acquisition to generate clinically useful images. The image quality of PET exams is often limited by patient motion during the exams. The lengthy scan times for imaging modalities such as PET may cause discomfort for patients and cause some movements. One solution to this issue is shortened or fast acquisition time. The direct result of shortening PET exam is that the corresponding image quality may be compromised. As another example, reduced radiation in CT may be achieved by lowering the operating current of the X-ray tube. Similar to PET, the reduced radiation may lead to reduced collected and detected photons which may in turn lead to increased noise in the reconstructed images. In another example, multiple pulse sequences (also known as image contrast) are usually acquired in MRI. In particular, Fluid-attenuated inversion recovery (FLAIR) sequence is commonly used to identify white matter lesions in the brain. However, when the FLAIR sequence is accelerated for a shorter scan time (similar to faster scan for PET), the small lesions are hard to be resolved.

### SUMMARY

Methods and systems are provided for enhancing quality of images such as medical images. The methods and systems provided herein may address various drawbacks of conventional systems, including those recognized above. Methods and systems provided herein may be capable of providing improved image quality with shortened image acquisition time, lower radiation dose, or reduced dose of tracer or contrast agent.

Methods and systems provided herein may allow for a faster and faster medical imaging without sacrificing image quality. Traditionally, short scan duration may result in low counts in the image frame and image reconstruction from the low-count projection data can be challenging due to that the tomography is ill-posed and of high noise. Furthermore, reducing the radiation dose may also lead to noisier images with degraded image quality. Methods and systems of described herein, may improve the quality of the medical image while preserving the quantification accuracy without modification to the physical system.

The provided methods and systems may significantly improve image quality by applying deep learning techniques so as to mitigate imaging artifacts and removing various types of noise. Examples of artifacts in medical imaging may include noise (e.g., low signal noise ratio), blur (e.g., motion artifact), shading (e.g., blockage or interference with sensing), missing information (e.g., missing pixels or voxels in painting due to removal of information or masking), and/or reconstruction (e.g., degradation in the measurement domain).

Additionally, methods and systems of the disclosure may be applied to existing systems without a need of a change of the underlying infrastructure. In particular, the provided methods and systems may accelerate PET scan time at no additional cost of hardware component and can be deployed regardless of the configuration or specification of the underlying infrastructure.

In an aspect, a computer-implemented method for improving image quality is provided. The method comprises: (a) acquiring, using a medical imaging apparatus, a medical image of a subject, wherein the medical image is acquired with shortened scanning time or reduced amount of tracer dose; and (b) applying a deep learning network model to the medical image to generate one or more attention feature maps and an enhanced medical image.

In a related yet separate aspect, a non-transitory computer-readable storage medium is provided including instructions that, when executed by one or more processors, cause the one or more processors to perform operations. The operations comprise: (a) acquiring, using a medical imaging apparatus, a medical image of a subject, wherein the medical image is acquired with shortened scanning time or reduced amount of tracer dose; and (b) applying a deep learning network model to the medical image to generate one or more attention feature maps and an enhanced medical image.

In some embodiments, the deep learning network model comprises a first subnetwork for generating the one or more attention feature maps and a second subnetwork for generating the enhanced medical image. In some cases, an input data to the second subnetwork includes the one or more attention feature maps. In some cases, the first subnetwork and the second subnetwork are deep learning networks. In some cases, the first subnetwork and the second subnetwork are trained in an end-to-end training process. In some instances, the second subnetwork is trained to adapt to the one or more attention feature maps.

In some embodiments, the deep learning network model includes a combination of U-net structure and a residual network. In some embodiments, the one or more attention feature maps include a noise map or lesion map. In some embodiments, the medical imaging apparatus is a transforming magnetic resonance (MR) device or a Positron Emission Tomography (PET) device.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of a workflow for processing and reconstructing medical image data, in accordance with some embodiments of the invention.

FIG. 1A illustrates an example of a Res-UNet model framework for producing a noise attention map or noise mask, in accordance with some embodiments of the invention.

FIG. 1B illustrates an example of Res-UNet model framework for adaptively enhancing image quality, in accordance with some embodiments of the invention.

FIG. 1C shows an example of a dual Res-UNets framework, in accordance with some embodiments of the invention.

FIG. 2 shows a block diagram of an exemplary PET image enhancement system, in accordance with embodiments of the disclosure.

FIG. 3 illustrates an example of method for improving image quality, in accordance with some embodiments of the invention.

FIG. 4 shows PET images taken under standard acquisition time, with accelerated acquisition, noise mask, and the enhance image processed by the provided methods and systems.

FIG. 5 schematically illustrates an example of the dual Res-UNets framework including a lesion attention subnetwork.

FIG. 6 shows an example lesion map.

FIG. 7 shows an example of a model architecture.

FIG. 8 shows an example of applying the deep learning self-attention mechanism to MR images.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides systems and methods that are capable of improving medical image quality. In particular, the provided systems and methods may employ a self-attention mechanism and adaptive deep learning framework that can significantly improve the image quality.

The provided systems and methods may improve image quality in various aspects. Examples of low quality in medical imaging may include noise (e.g., low signal noise ratio), blur (e.g., motion artifact), shading (e.g., blockage or interference with sensing), missing information (e.g., missing pixels or voxels in painting due to removal of information or masking), reconstruction (e.g., degradation in the measurement domain), and/or under-sampling artifacts (e.g., under-sampling due to compressed sensing, aliasing).

In some cases, the provided systems and methods may employ a self-attention mechanism and adaptive deep learning framework to improve the image quality of low-dose Positron Emission Tomography (PET) or fast-scanned PET and achieve high quantification accuracy. Positron Emission Tomography (PET) is a nuclear medicine functional imaging technique that is used to observe metabolic processes in the body as an aid to the diagnosis of disease. A PET system may detect pairs of gamma rays emitted indirectly by a positron-emitting radioligand, most commonly fluorine-18, which is introduced into a patient body on a biologically active molecule such as a radioactive tracer. The biologically active molecule can be any suitable type such as fludeoxyglucose (FDG). With tracer kinetic modeling, PET is capable of quantifying physiologically or biochemically important parameters in regions of interest or voxel-wise to detect disease status and characterize severity.

Though positron emission tomography (PET) and PET data examples are primarily provided herein, it should be understood that the present approach may be used in other imaging modality contexts. For instance, the presently described approach may be employed on data acquired by other types of tomographic scanners including, but not limited to, computed tomography (CT), single photon emission computed tomography (SPECT) scanners, functional magnetic resonance imaging (fMRI), or magnetic resonance imaging (MRI) scanners.

The term “accurate quantification” or “quantification accuracy” of PET imaging may refer to the accuracy of quantitative biomarker assessment such as radioactivity distribution. Various metrics can be employed for quantifying the accuracy of PET image such as standardized uptake value (SUV) for an FDG-PET scan. For example, peak SUV value may be used as metric for quantifying accuracy of the PET image. Other common statistics such as mean, median, min, max, range, skewness, kurtosis, and more complex values, such as metabolic volume above an absolute SUV of 5 standardized uptake value (SUV) of 18-FDG, can also be calculated and used for quantifying the accuracy of PET imaging.

The term “shortened acquisition,” as used herein, generally refers to shortened PET acquisition time or PET scan duration. The provided systems and methods may be able to achieve PET imaging with improved image quality by an acceleration factor of at least 1.5, 2, 3, 4, 5, 10, 15, 20, a factor of a value above 20 or below 1.5, or a value between any of the two aforementioned values. An accelerated acquisition can be achieved by shortening the scan duration of a PET scanner. For example, an acquisition parameter (e.g., 3 min/bed, 18 min in total) may be set up via the PET system prior to performing a PET scan.

1. The provided systems and methods may allow for a faster and safer PET acquisition. As described above, PET images taken under short scan duration and/or reduced radiation dose may have low image quality (e.g., high noise) due to low coincident-photon counts detected in addition to various physical degradation factors. Example of sources of noise in PET may include scatter (a detected pair of photons, at least one of which was deflected from its original path by interaction with matter in the field of view, leading to the pair being assigned to an incorrect line-of-response) and random events (photons originating from two different annihilation events but incorrectly recorded as a coincidence pair because their arrival at their respective detectors occurred within a coincidence timing window. Methods and systems of described herein, may improve the quality of the medical image while preserving the quantification accuracy without modification to the physical system.

Methods and systems provided herein may further improve the acceleration capability of imaging modalities over existing acceleration methods by utilizing a self-attention deep learning mechanism. In some embodiments, the self-attention deep learning mechanism may be capable of identifying regions of interest (ROI) such as lesions or areas containing pathology on the images, and an adaptive deep learning enhancement mechanism may be used to further optimize the image quality within the ROIs. In some embodiments, the self-attention deep learning mechanism and the adaptive deep learning enhancement mechanism may be implemented by a dual Res-UNets framework. The dual Res-UNets framework may be designed and trained to identify features that highlighting the region-of-interest (ROI) in the low-quality PET images first, then incorporate the ROI attention information to perform image enhancement and obtain high-quality PET images.

Methods and systems provided herein may be capable of reducing noise of the image regardless the distribution of the noise, characteristics of the noise or the types of modalities. For instance, noise in medical images may not be distributed evenly. Methods and systems provided herein may resolve the mixed noise distribution in low quality image, by implementing a general and adaptive robust loss mechanism which may automatically fit the model training to learn the optimal loss. The general and adaptive robust loss mechanism may also beneficially adapt to different modalities. In the case of PET, PET images may suffer from artifacts that may include noise (e.g., low signal noise ratio), blur (e.g., motion artifact), shading (e.g., blockage or interference with sensing), missing information (e.g., missing pixels or voxels in painting due to removal of information or masking), reconstruction (e.g., degradation in the measurement domain), sharpness and various other artifacts that may lower the quality of the image. In addition to the accelerated acquisition factor, other sources may also introduce noise in PET imaging which may include scatter (a detected pair of photons, at least one of which was deflected from its original path by interaction with matter in the field of view, leading to the pair being assigned to an incorrect LOR) and random events (photons originating from two different annihilation events but incorrectly recorded as a coincidence pair because their arrival at their respective detectors occurred within a coincidence timing window). In the case of MRI images, the input images may suffer from noise such as salt and pepper noise, speckle noise, Gaussian noise and Poisson noise or other artifact such motion or breathing artifact. The self-attention deep learning mechanism and the adaptive deep learning enhancement mechanism may automatically identify ROIs and optimize the image enhancement within the ROIs regardless the types of image. The improved data fitting mechanism may result in better image enhancement and provide an improved denoising result.

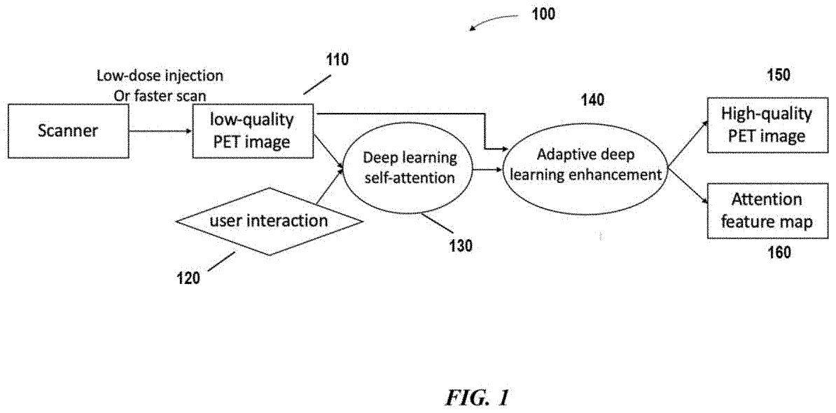

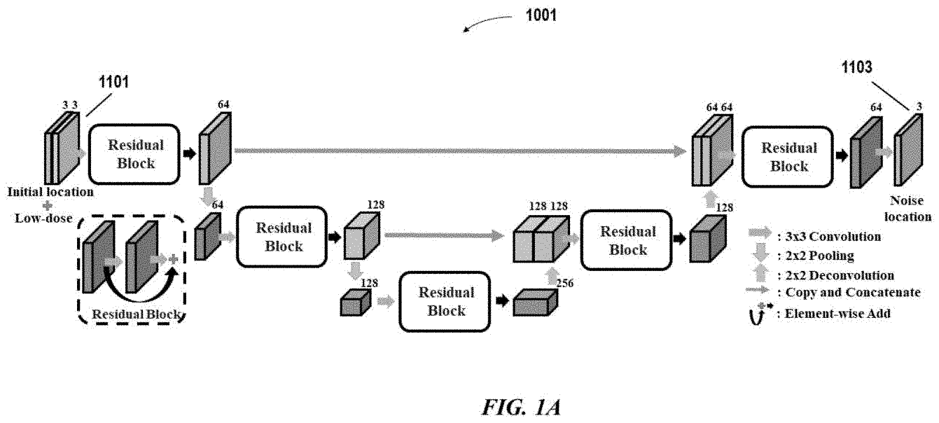

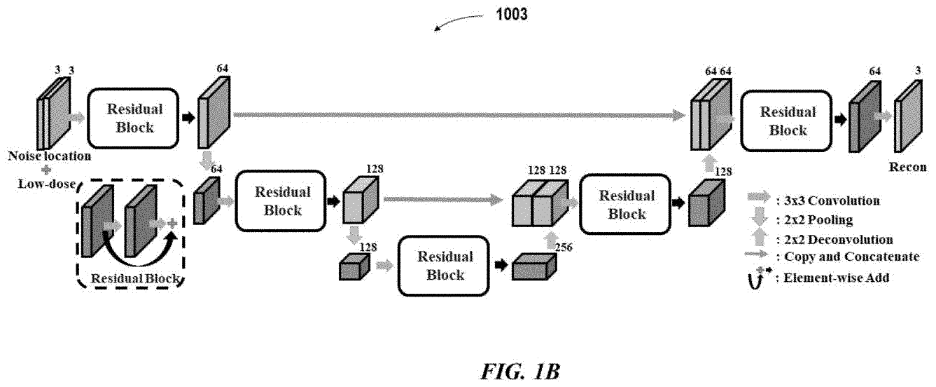

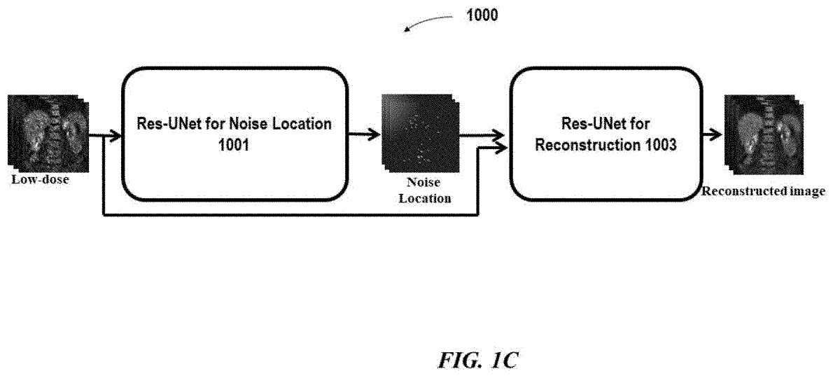

FIG. 1 shows an example of a workflow 100 for processing and reconstructing image data. The images may be obtained from any medical imaging modality such as but not limited to CT, fMRI, SPECT, PET, ultrasound, etc. Image quality may be degraded due to for example fast acquisition or reduction in radiation dose or presence of noise in imaging sequence. The acquired images 110 may be low-quality image such as low resolution or low signal to noise ratio (SNR). For example, the acquired images may be PET images 101 with low image resolution and/or signal to noise ratio (SNR) due to fast acquisition or reduction in radiation dose (e.g., radiotracer) as described above.

The PET images 110 may be acquired by complying with an existing or conventional scan protocol such as metabolic volume calibration or interinstitutional cross-calibration and quality control. The PET images 110 may be acquired and reconstructed using any conventional reconstruction techniques without additional change to the PET scanner. The PET images 110 acquired with shortened scan duration may also be referred to as low-quality image or original input image which can be used interchangeably throughout the specification.

In some cases, the acquired images 110 may be reconstructed image obtained using any existing reconstruction method. For example, the acquired PET images may be reconstructed using filtered back projection, statistical, likelihood-based approaches, and various other conventional methods. However, the reconstructed images may still have low image quality such as low resolution and/or low SNR due to the shortened acquisition time and reduced number of detected photons. The acquired images 110 may be 2D image data. In some cases, the input data may be 3D volume comprising multiple axial slices.

Image quality of the low resolution images may be improved using a serialized deep learning system. The serialized deep learning system may comprise a deep learning self-attention mechanism 130 and an adaptive deep learning enhancement mechanism 140. In some embodiments, the input to the serialized deep learning system may be low-quality image 110 and the output may be the corresponding high-quality image 150.

In some embodiments, the serialized deep learning system may receive user input 120 related to the ROI and/or user preferred output result. For instance, a user may be permitted to set enhancement parameters or identify regions of interest (ROI) in the lower quality images to be enhanced. In some cases, a user may be able to interact with the system to select a target goal of the enhancement (e.g., reduce noise of entire image or in a selected ROI, generate pathology information in a user-selected ROI, etc). As a non-limiting example, if users choose to enhance the low-quality PET image with extreme noise (e.g., high-intensity noise), the system may focus on distinguishing the high-intensity noise and pathology and improve the overall image quality, the output of the system may be an image with improved quality. If users choose to enhance the image quality of specific ROIs (e.g., tumors), the system may output ROI probability map highlighting the ROI location and the high-quality PET image 150. The ROI probability map may be an attention feature map 160.

The deep learning self-attention mechanism 130 may be a trained deep learning model that is capable of detecting the desired ROIs attention. The model network may be a deep learning neural network designed to apply a self-attention mechanism on the input images (e.g., low quality image). The self-attention mechanism may be used for segmentation of image and identification of ROIs. The self-attention mechanism may be a trained model that is able to identify features that corresponding to the region-of-interest (ROI) in the low-quality PET images. For example, the deep learning self-attention mechanism may be trained to be able to distinguish between high-intensity small abnormality and high-intensity noise, i.e., extreme noise. In some cases, the self-attention mechanism may identify the desired ROIs attention automatically.

The region-of-interest (ROI) may be region where extreme noise located or a region of diagnostic region of interest. The ROIs attention may be noise attention or clinically-meaningful attention (e.g., lesion attention, pathology attention, etc). The noise attention may comprise information such as noise location in the input low-quality PET image. The ROIs attention may be the lesion attention that need more accurate boundary enhancement compared to the normal structures and background. For CT images, the ROIs attention may be a metal region attention that the provided model framework is capable of distinguishing between bone structure and metal structure.

In some embodiments, the input of the deep learning self-attention model 130 may comprise low-quality image data 110, and the output of the deep learning self-attention model 130 may comprise an attention map. The attention map may comprise an attention feature map or ROI attention masks. The attention map may be a noise attention map that comprises information about the location of noise (e.g., coordinates, distribution, etc), a lesion attention map or other attention map that comprises clinically meaningful information. For example, the attention map for CT may comprise information about a metal region in the CT images. In another example, the attention map may comprise information about regions where particular tissues/features are located.

As described elsewhere herein, the deep learning self-attention model 130 may identify the ROIs and provide an attention feature map such as a noise mask. In some cases, the output of the deep learning self-attention model may be a set of ROI attention masks that indicate the regions require further analysis, which may be inputted to the adaptive deep learning enhancement module to achieve high-quality images (e.g., accurate high-quality PET image 150). The ROI attention masks may be pixel-wise masks or voxel-wise masks.

In some cases, the ROI attention masks or attention feature map may be produced using segmentation techniques. For instance, ROI attention masks such as noise mask may occupy a small portion of the entire image which may cause a class imbalance between candidate labels in the labeling process. In order to avoid the imbalance strategies such as but not limited to weighted cross-entropy function, the sensitivity function or the Dice loss function may be used to determine accurate ROI segmentation result. Binary cross entropy loss may also be used to stabilize the training of the deep learning ROI detection network.

The deep learning self-attention mechanism may comprise a trained model for producing ROI attention masks or attention feature map. As an example, the deep learning neural network may be trained for noise detection with the noise attention as foreground. As described elsewhere, the foreground of the noise mask may only occupy a small percentage of the entire image, which may create a typical class imbalance problem. In some cases, a Dice loss ( [symbol] _{DICE}) may be utilized as the loss function to overcome this problem. In some cases, a binary cross entropy loss ( [symbol] _{BCE}) may be used to form the voxel-wise measurement to stabilize the training process. The total loss ( [symbol] _{Atten}) for noise-attention can be formulated as follows:

$${ℒ}_{DICE} ( ρ , \hat{ρ} ) = 1 - \frac{2 〈 ρ , \hat{ρ} 〉}{{‖ ρ ‖}_{2}^{2} + {‖ \hat{ρ} ‖}_{2}^{2}} \\ {ℒ}_{BCE} ( ρ , \hat{ρ} ) = - ( ρ log ( \hat{ρ} ) + ( 1 - ρ ) log ( 1 - \hat{ρ} ) ) \\ {ℒ}_{Atten} = {ℒ}_{BCE} + α {ℒ}_{DICE}$$

where ρ represents the ground-truth data such as the full-dose or standard time PET image or full dose radiation CT image, etc, \hat{ρ} represents the reconstructed result by the proposed image enhancement method, and α represents the weight that balances [symbol] _{BCE} and [symbol] _{DICE}.

The deep learning self-attention model can employ any type of neural network model, such as a feedforward neural network, radial basis function network, recurrent neural network, convolutional neural network, deep residual learning network and the like. In some embodiments, the machine learning algorithm may comprise a deep learning algorithm such as convolutional neural network (CNN). The model network may be a deep learning network such as CNN that may comprise multiple layers. For example, the CNN model may comprise at least an input layer, a number of hidden layers and an output layer. A CNN model may comprise any total number of layers, and any number of hidden layers. The simplest architecture of a neural network starts with an input layer followed by a sequence of intermediate or hidden layers, and ends with output layer. The hidden or intermediate layers may act as learnable feature extractors, while the output layer may output the noise mask or a set of ROI attention masks. Each layer of the neural network may comprise a number of neurons (or nodes). A neuron receives input that comes either directly from the input data (e.g., low quality image data, fast-scanned PET data, etc.) or the output of other neurons, and performs a specific operation, e.g., summation. In some cases, a connection from an input to a neuron is associated with a weight (or weighting factor). In some cases, the neuron may sum up the products of all pairs of inputs and their associated weights. In some cases, the weighted sum is offset with a bias. In some cases, the output of a neuron may be gated using a threshold or activation function. The activation function may be linear or non-linear. The activation function may be, for example, a rectified linear unit (ReLU) activation function or other functions such as saturating hyperbolic tangent, identity, binary step, logistic, arcTan, softsign, parameteric rectified linear unit, exponential linear unit, softPlus, bent identity, softExponential, Sinusoid, Sinc, Gaussian, sigmoid functions, or any combination thereof.

In some embodiments, the self-attention deep learning model may be trained using supervised learning. For example, in order to train the deep learning network, pairs of fast-scanned PET images with low quality (i.e., acquired under reduced time or lower radiotracer dosage) and standard/high quality PET images as ground truth from multiple subjects may be provided as training dataset.

In some embodiments, the model may be trained using unsupervised learning or semi-supervised learning that may not require abundant labeled data. High quality medical image datasets or paired dataset can be hard to collect. In some cases, the provided method may utilize unsupervised training approach allowing the deep learning method to train and apply on existing datasets (e.g., unpaired dataset) that are already available in clinical database.

In some embodiments, the training process of the deep learning model may employ residual learning method. In some cases, the network structure can be a combination of U-net structure and a residual network. FIG. 1A illustrates an example of a Res-UNet model framework 1001 for identifying noise attention map or generating a noise mask. A Res-UNet is an extension of UNet with residual blocks in each resolution stage. The Res-UNet model framework takes advantage of two network architectures, UNet and Res-Net. The illustrated Res-UNet 1001 takes low-dose PET image as input 1101 and generates a noise attention probability map or noise mask 1103. As shown in the example, the Res-UNet architecture comprises 2 pooling layers, 2 upsampling layers and 5 residual blocks. The Res-UNet architecture can have any other suitable forms (e.g., different number of layers) according to different performance requirement.

Referring back to FIG. 1, the ROI attention masks or attention feature maps may be passed on to an adaptive deep learning enhancement network 140 for enhancing image quality. In some cases, the ROI attention masks such as noise feature map may be concatenated with the original low-dose/fast-scanned PET image and passed on to the adaptive deep learning enhancement network for image enhancement.

In some embodiments, the adaptive deep learning network 140 (e.g., Res-UNet) may be trained to enhance the image quality and perform adaptive image enhancement. As described above, the input to the adaptive deep learning network 140 may comprise the low-quality image 110 and the output generated by the deep-learning self-attention network 130 such as the attention feature map or the ROI attention masks (e.g., noise mask, lesion attention map). The output of the adaptive deep learning network 140 may comprise high-quality/denoised images 150. Optionally, an attention feature map 160 may also be generated and presented to the user. The attention feature map 160 can be the same as the attention feature map supplied to the adaptive deep learning network 140. Alternatively, the attention feature map 160 may be produced based on the output of the deep learning self-attention network and presented in a form (e.g., heat map, color diagram, etc) that is easily comprehended by a user such as a noise attention probability map.

The adaptive deep learning network 140 may be trained to be capable of adapting to various noise distributions (e.g., Gaussian, Poisson, etc). The adaptive deep learning network 140 and the deep-learning self-attention network 130 may be trained in an end-to-end training process such that the adaptive deep learning network 140 can adapt to various types of noise distributions. For example, by implementing the adaptive robust loss mechanism (loss function), the parameters of the deep-learning self-attention network may be tuned automatically to fit the model to learn the optimal total loss by adaptive to the attention feature maps.

In the end-to-end training process, in order to automatically adapt to the distribution of various types of noise in the images such as Gaussian noise or Poisson noise, a general and adaptive robust loss may be designed to fit the noise distribution of the input low-quality image. The general and adaptive robust loss may be applied to automatically determine the loss function during training without manual parameter tuning. This approach may beneficially adjust the optimal loss function according to the data (e.g., noise) distribution. Below is an example of the loss function:

$${ℒ}_{GAR} ( ρ , \hat{ρ} ) = \frac{\vert α - 2 \vert}{α} ( {( \frac{{( \frac{ρ - \hat{ρ}}{c} )}^{2}}{\vert α - 2 \vert} + 1 )}^{\frac{α}{2}} - 1 )$$

where α and c are two parameters that need to be learned during training, the first one controls the robustness of the loss and the second one controls the size of the loss's near ρ−\hat{β}=0. ρ represents the ground-truth data such as the full-dose or standard time PET image or full dose radiation CT image, etc and \hat{ρ} represents the reconstructed result by the proposed image enhancement method.

In some embodiments, the adaptive deep learning network may employ residual learning method. In some cases, the network structure can be a combination of U-net structure and a residual network. FIG. 1B illustrates an example of a Res-UNet model framework 1003 for adaptively enhancing image quality. The illustrated Res-UNet 1003 may take the low-quality image and the output of the deep-learning self-attention network 130 such as the attention feature map or the ROI attention masks (e.g., noise mask, lesion attention map) as the input, and output the high-quality image corresponding to the low-quality image. As shown in the example, the Res-UNet architecture comprises 2 pooling layers, 2 upsampling layers and 5 residual blocks. The Res-UNet architecture can have any other suitable forms (e.g., different number of layers) according to different performance requirement.

The adaptive deep learning network can employ any type of neural network model, such as a feedforward neural network, radial basis function network, recurrent neural network, convolutional neural network, deep residual learning network and the like. In some embodiments, the machine learning algorithm may comprise a deep learning algorithm such as convolutional neural network (CNN). The model network may be a deep learning network such as CNN that may comprise multiple layers. For example, the CNN model may comprise at least an input layer, a number of hidden layers and an output layer. A CNN model may comprise any total number of layers, and any number of hidden layers. The simplest architecture of a neural network starts with an input layer followed by a sequence of intermediate or hidden layers, and ends with output layer. The hidden or intermediate layers may act as learnable feature extractors, while the output layer may generate high-quality image. Each layer of the neural network may comprise a number of neurons (or nodes). A neuron receives input that comes either directly from the input data (e.g., low quality image data, fast-scanned PET data, etc.) or the output of other neurons, and performs a specific operation, e.g., summation. In some cases, a connection from an input to a neuron is associated with a weight (or weighting factor). In some cases, the neuron may sum up the products of all pairs of inputs and their associated weights. In some cases, the weighted sum is offset with a bias. In some cases, the output of a neuron may be gated using a threshold or activation function. The activation function may be linear or non-linear. The activation function may be, for example, a rectified linear unit (ReLU) activation function or other functions such as saturating hyperbolic tangent, identity, binary step, logistic, arcTan, softsign, parameteric rectified linear unit, exponential linear unit, softPlus, bent identity, softExponential, Sinusoid, Sinc, Gaussian, sigmoid functions, or any combination thereof.

In some embodiments, the model for enhancing image quality may be trained using supervised learning. For example, in order to train the deep learning network, pairs of fast-scanned PET images with low quality (i.e., acquired under reduced time) and standard/high quality PET images as ground truth data from multiple subjects may be provided as training dataset.

In some embodiments, the model may be trained using unsupervised learning or semi-supervised learning that may not require abundant labeled data. High quality medical image datasets or paired dataset can be hard to collect. In some cases, the provided method may utilize unsupervised training approach allowing the deep learning method to train and apply on existing datasets (e.g., unpaired dataset) that are already available in clinical database. In some embodiments, the training process of the deep learning model may employ residual learning method. In some cases, the network structure can be a combination of U-net structure and a residual network.

In some embodiments, the provided deep learning self-attention mechanism and adaptive deep learning enhancement mechanism may be implemented using a dual Res-UNets framework. The dual Res-UNets framework may be a serialized deep learning framework. The deep learning self-attention mechanism and adaptive deep learning enhancement mechanism may be sub-networks of the dual Res-UNets framework. FIG. 1C shows an example of the dual Res-UNets framework 1000. In the illustrated example, the dual Res-UNets framework may comprise a first sub-network which is a Res-UNet 1001 configured for automatically identifying ROI attention in the input image (e.g., low-quality image). The first sub-network (Res-UNet) 1001 can be the same as the network as described in FIG. 1A. The output of the first sub-network (Res-UNet) 1001 may be combined with the original low-quality image and transferred to the second sub-network which can be a Res-UNet 1003. The second sub-network (Res-UNet) 1003 can be the same as the network as described in FIG. 1B. The second sub-network (Res-UNet) 1003 may be trained to generate a high-quality image.

In preferred embodiments, the two sub-networks (Res-UNets) may be trained as an integral system. For instance, during an end-to-end training, the loss for training the first Res-UNet and the loss for training the second Res-UNet may be summed to reach a total loss for training the integral deep learning network or system. The total loss may be a weighted sum of the two losses. In other cases, the output of the first Res-UNet 1001 may be used for training the second Res-UNet 1003. For example, the noise mask generated by the first Res-UNet 1001 may be used as part of the input feature for training the second Res-UNet 1003.

Methods and system described herein can be applied to other modality image enhancement, such as but not limited to lesion enhancement in MRI image and metal removal in CT image. For example, for lesion enhancement in MRI mage, the deep learning self-attention module may generate the lesion attention mask first, and the adaptive deep learning enhancement module may enhance the lesion in the identified region according to the attention map. In another example, for CT images, it may be difficult to distinguish between bone structures and metal structure since the may share same image featured such as intensity value. Methods and systems described herein may accurately distinguish bone structure from metal structure using the deep learning self-attention mechanism. The metal structure may be identified on an attention feature map. The adaptive deep learning mechanism may use the attention feature map to remove the unwanted structures in the image.

### System Overview

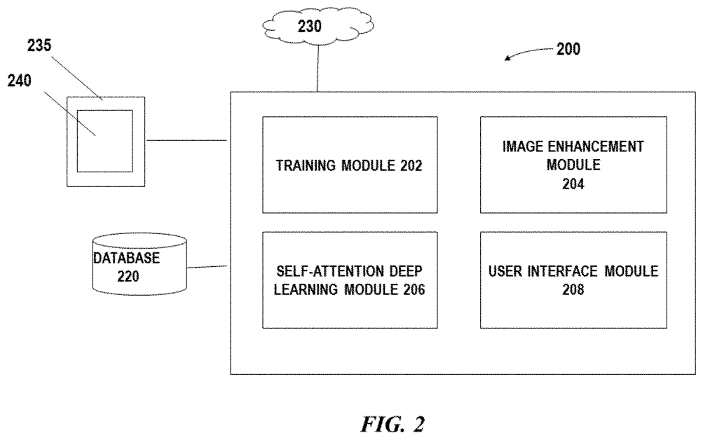

The systems and methods can be implemented on existing imaging systems such as but not limited to PET imaging systems without a need of a change of hardware infrastructure. FIG. 2 schematically illustrates an example PET system 200 comprising a computer system 210 and one or more databases operably coupled to a controller over the network 230. The computer system 210 may be used for further implementing the methods and systems explained above to improve the quality of images.

The controller 201 (not shown) may be a coincidence processing unit. The controller may comprise or be coupled to an operator console (not shown) which can include input devices (e.g., keyboard) and control panel and a display. For example, the controller may have input/output ports connected to a display, keyboard and printer. In some cases, the operator console may communicate through the network with a computer system that enables an operator to control the production and display of images on a screen of display. The images may be images with improved quality and/or accuracy acquired according to an accelerated acquisition scheme. The image acquisition scheme may be determined automatically by the PET imaging accelerator and/or by a user as described later herein.

The PET system may comprise a user interface. The user interface may be configured to receive user input and output information to a user. The user input may be related to controlling or setting up an image acquisition scheme. For example, the user input may indicate scan duration (e.g., the min/bed) for each acquisition or scan time for a frame that determines one or more acquisition parameters for an accelerated acquisition scheme. The user input may be related to the operation of the PET system (e.g., certain threshold settings for controlling program execution, image reconstruction algorithms, etc). The user interface may include a screen such as a touch screen and any other user interactive external device such as handheld controller, mouse, joystick, keyboard, trackball, touchpad, button, verbal commands, gesture-recognition, attitude sensor, thermal sensor, touch-capacitive sensors, foot switch, or any other device.

The PET imaging system may comprise computer systems and database systems 220, which may interact with a PET imaging accelerator. The computer system may comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, or a plurality of processors for parallel processing. The processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations. The imaging platform may comprise one or more databases. The one or more databases 220 may utilize any suitable database techniques. For instance, structured query language (SQL) or “NoSQL” database may be utilized for storing image data, raw collected data, reconstructed image data, training datasets, trained model (e.g., hyper parameters), adaptive mixing weighting coefficients, etc. Some of the databases may be implemented using various standard data-structures, such as an array, hash, (linked) list, struct, structured text file (e.g., XML), table, JSON, NOSQL and/or the like. Such data-structures may be stored in memory and/or in (structured) files. In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present disclosure is implemented as a data-structure, the use of the database of the present disclosure may be integrated into another component such as the component of the present disclosure. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated.

The network 230 may establish connections among the components in the imaging platform and a connection of the imaging system to external systems. The network 230 may comprise any combination of local area and/or wide area networks using both wireless and/or wired communication systems. For example, the network 230 may include the Internet, as well as mobile telephone networks. In one embodiment, the network 230 uses standard communications technologies and/or protocols. Hence, the network 230 may include links using technologies such as Ethernet, 802.11, worldwide interoperability for microwave access (WiMAX), 2G/3G/4G mobile communications protocols, asynchronous transfer mode (ATM), InfiniBand, PCI Express Advanced Switching, etc. Other networking protocols used on the network 230 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), and the like. The data exchanged over the network can be represented using technologies and/or formats including image data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS), Internet Protocol security (IPsec), etc. In another embodiment, the entities on the network can use custom and/or dedicated data communications technologies instead of, or in addition to, the ones described above.

The imaging platform may comprise multiple components, including but not limited to, a training module 202, an image enhancement module 204, a self-attention deep learning module 206 and a user interface module 208.

The training module 202 may be configured to train a serialized machine learning model framework. The training module 202 may be configured to train a first deep learning model for identifying ROI attention and a second model for adaptively enhancing image quality. The training module 202 may train the two deep learning models separately. Alternatively or in addition to, the two deep learning models may be trained as an integral model.

The training module 202 may be configured to obtain and manage training datasets. For example, the training datasets for the adaptive image enhancement may comprise pairs of standard acquisition and shortened acquisition images and/or attention feature map from same subject. The training module 202 may be configured to train a deep learning network for enhancing the image quality as described elsewhere herein. For example, the training module may employ supervised training, unsupervised training or semi-supervised training techniques for training the model. The training module may be configured to implement the machine learning methods as described elsewhere herein. The training module may train a model off-line. Alternatively or additionally, the training module may use real-time data as feedback to refine the model for improvement or continual training.

The image enhancement module 204 may be configured to enhance image quality using a trained model obtained from the training module. The image enhancement module may implement the trained model for making inferences, i.e., generating PET images with improved quality.

The self-attention deep learning module 206 may be configured to generate ROI attention information such attention feature map or ROI attention masks using a trained model obtained from the training module. The output of the self-attention deep learning module 206 may be transmitted to the image enhancement module 204 as part of the input to the image enhancement module 204.

The computer system 200 may be programmed or otherwise configured to manage and/or implement an enhanced PET imaging system and its operations. The computer system 200 may be programmed to implement methods consistent with the disclosure herein.

The computer system 200 may include a central processing unit (CPU, also “processor” and “computer processor” herein), a graphic processing unit (GPU), a general-purpose processing unit, which can be a single core or multi core processor, or a plurality of processors for parallel processing. The computer system 200 can also include memory or memory location (e.g., random-access memory, read-only memory, flash memory), electronic storage unit (e.g., hard disk), communication interface (e.g., network adapter) for communicating with one or more other systems, and peripheral devices 235, 220, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 200 can be operatively coupled to a computer network (“network”) 230 with the aid of the communication interface. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 200, can implement a peer-to-peer network, which may enable devices coupled to the computer system 200 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system 200 in some cases can include one or more additional data storage units that are external to the computer system, such as located on a remote server that is in communication with the computer system through an intranet or the Internet.

The computer system 200 can communicate with one or more remote computer systems through the network 230. For instance, the computer system 200 can communicate with a remote computer system of a user or a participating platform (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 300 via the network 230.

Methods as described herein can be implemented by way of machine (e.g., computer processor) executable code stored on an electronic storage location of the computer system 200, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 200 can include or be in communication with an electronic display 235 that comprises a user interface (UI) for providing, for example, displaying reconstructed images or acquisition speeds. Examples of UI's include, without limitation, a graphical user interface (GUI) and web-based user interface.

The system 200 may comprise a user interface (UI) module 208. The user interface module may be configured to provide a UI to receive user input related to the ROI and/or user preferred output result. For instance, a user may be permitted to set enhancement parameters or identify regions of interest (ROI) in the lower quality images to be enhanced via the UI. In some cases, a user may be able to interact with the system via the UI to select a target goal of the enhancement (e.g., reduce noise of entire image or in ROI, generate pathology information in a user-selected ROI, etc). The UI may display the improved image and/or a ROI probability map (e.g., noise attention probability mal).

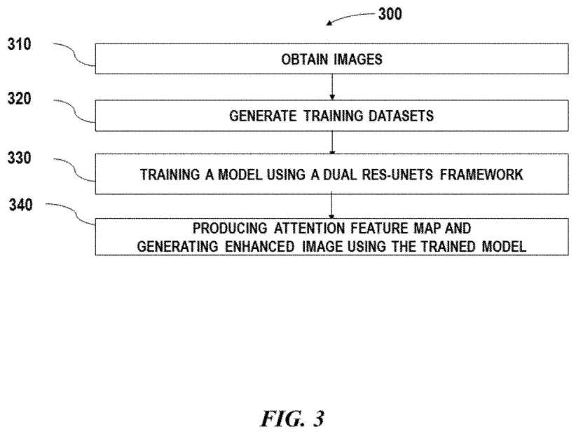

Methods and systems of the present disclosure can be implemented by way of one or more algorithms. An algorithm can be implemented by way of software upon execution by the central processing unit. For example, some embodiments may use the algorithm illustrated in FIG. 1 and FIG. 3 or other algorithms provided in the associated descriptions above.

FIG. 3 illustrates an exemplary process 300 for improving image quality from low resolution or noisy images. A plurality of images may be obtained from a medical imaging system such as PET imaging system (operation 310) for training a deep learning model. The plurality of PET images for forming a training dataset 320 can also be obtained from external data sources (e.g., clinical database, etc.) or from simulated image sets. In a step 330, a dual residual-Unet framework is used to train a model based on the training datasets. The dual residual-Unet framework may include for example, a self-attention deep learning model as described elsewhere herein that is used for generating an attention feature map (e.g., ROI map, noise mask, lesion attention map, etc.) and a second deep learning mechanism may be used to adaptively enhance the quality of images. In a step 340, a trained model may be deployed to make predictions to enhance the image quality.

### Example Dataset

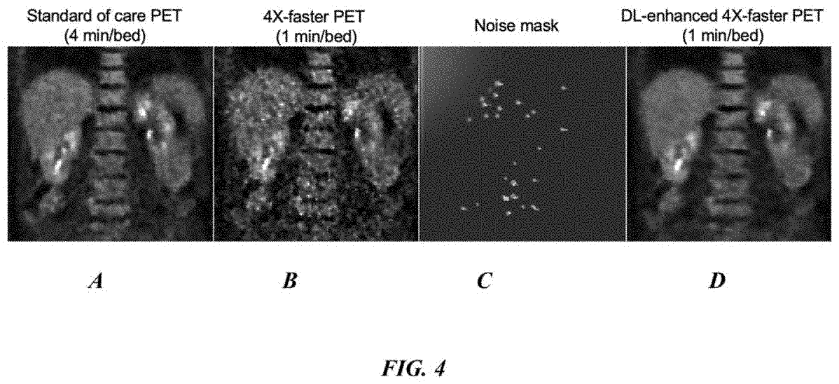

FIG. 4 shows PET images taken under standard acquisition time (A), with accelerated acquisition (B), noise mask produce by the deep learning attention mechanism C and the fast-scanned image processed by the provided methods and systems (D). A shows a standard PET image with no enhancement or shortened acquisition time. The acquisition time for this example is 4 minutes per bed (min/bed). This image may be used in training the deep learning network as an example of the ground truth. A shows an example of a PET image with shortened acquisition time. In this example the acquisition time is accelerated by 4 times and the acquisition time is reduced to 1 min/bed. The fast-scanned image present lower image quality such as high noise. This image may be an example of the second image used in pairs of images for training the deep learning network along with the generated noise mask C from these two images. D shows an example of an improved quality image which the methods and systems of the present disclosure are applied to. The image quality has substantially improved and comparable to the standard PET image quality.

### Example

In one study, ten subjects (age: 57±16 years, weight: 80±17 Kgs) referred for a whole-body FDG-18 PET/CT scan on a GE Discovery scanner (GE Healthcare, Waukesha, Wis.) were recruited for this study following IRB approval and informed consent. The standard of care was a 3.5 min/bed PET acquisition acquired in list-mode. 4-fold dose reduction PET acquisitions were synthesized as the low-dose PET image using the list-mode data from the original acquisitions. Quantitative image quality metrics such as normalized root-mean-squared-error (NRMSE), peak signal to noise ratio (PSNR), and structural similarity (SSIM) were calculated for all enhanced and non-enhanced accelerated PET scans, with the standard 3.5 min acquisition as the ground-truth. The results are shown in Table. 1. Better image quality is achieved using the proposed system.

TABLE 1
Results of image quality metrics

| | NRMSE | PSNR | SSIM |
|---|---|---|---|
| Non-Enhanced | 0.69 ± 0.15 | 50.52 ± 4.38 | 0.87 ± 0.43 |
| DL-Enhanced | 0.63 ± 0.12 | 53.66 ± 2.61 | 0.91 ± 0.25 |

### MRI Example

The presently described approach may be employed on data acquired by a variety types of tomographic scanners including, but not limited to, computed tomography (CT), single photon emission computed tomography (SPECT) scanners, functional magnetic resonance imaging (fMRI), or magnetic resonance imaging (MRI) scanners. In MRI multiple pulse sequences (also known as image contrast) are usually acquired. For example, Fluid-attenuated inversion recovery (FLAIR) sequence is commonly used to identify white matter lesions in the brain. However, when the FLAIR sequence is accelerated for a shorter scan time (similar to faster scan for PET), the small lesions are hard to be resolved. The self-attention mechanism and adaptive deep learning framework as described herein can also be easily applied in MRI to enhance the image quality.

In some cases, the self-attention mechanism and adaptive deep learning framework may be applied to accelerate MRI by enhancing quality of the raw images that have low image quality such as low resolution and/or low SNR due to the shortened acquisition time. By employing the self-attention mechanism and adaptive deep learning framework, MRI can be performed with faster scanning while remaining high quality reconstruction.

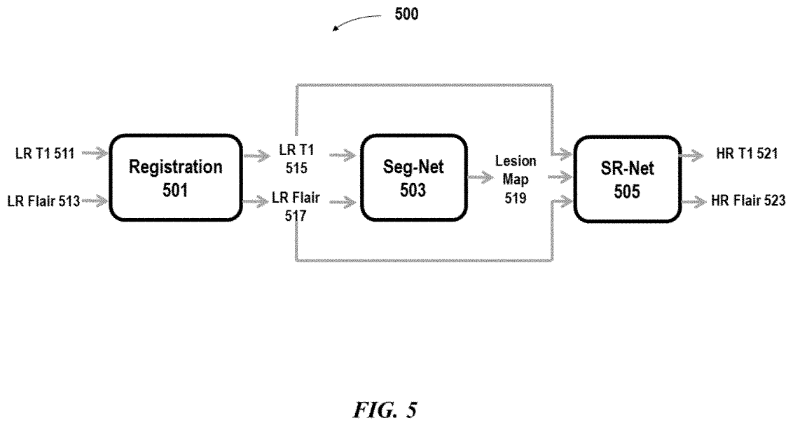

As described above, the region-of-interest (ROI) may be region where extreme noise located or a region of diagnostic region of interest. The ROIs attention may be the lesion attention that need more accurate boundary enhancement compared to the normal structures and background. FIG. 5 schematically illustrates an example of the dual Res-UNets framework 500 including a lesion attention subnetwork. Similar to the framework as described in FIG. 1C, the dual Res-UNets framework 500 may include a segmentation-Net 503 and an adaptive deep learning subnetwork 505 (Super-resolution network (SR-net)). In the illustrated example, the segmentation-Net 503 may be a subnetwork trained to perform lesion segmentation (e.g., white matter lesion segmentation) and the output of the segmentation-Net 503 may include a lesion map 519. The lesion map 519 and low quality images may then be processed by the adaptive deep learning subnetwork 505 to produce high quality images (e.g., high-resolution T1 521, high-resolution FLAIR 523).

The segmentation-Net 503 may receive the input data with low quality (e.g., low resolution T1 511 and low-resolution FLAIR images 513). The low-resolution T1 and low-resolution FLAIR images may be registered 501 using a registration algorithm to form a pair of registered images 515, 517. For example, image/volume co-registration algorithms may be applied to generate spatially matched images/volumes. In some cases, the co-registration algorithms may comprise a coarse scale rigid algorithm to achieve an initial estimation of an alignment, followed by a fine-grain rigid/non-rigid co-registration algorithm.

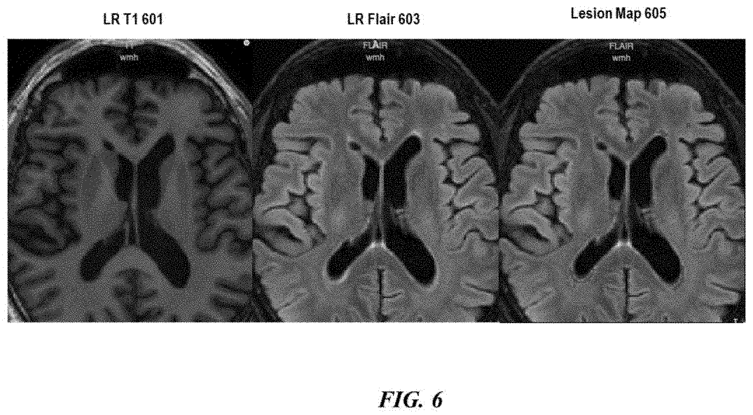

Next, the registered low resolution T1 and low resolution FLAIR images may be received by the segmentation-Net 503 to output a lesion map 519. FIG. 6 shows an example of a pair of registered low-resolution T1 images 601 and low-resolution FLAIR images 603 as well as a lesion map 605 superimposed on the image.

Referring back to FIG. 5, The registered low resolution T1 images 515, low resolution FLAIR images 517 as well as lesion map 519 may then be processed by the deep learning subnetwork 505 to output the high-quality MR images (e.g., high-resolution T1 521 and high-resolution FLAIR 523).

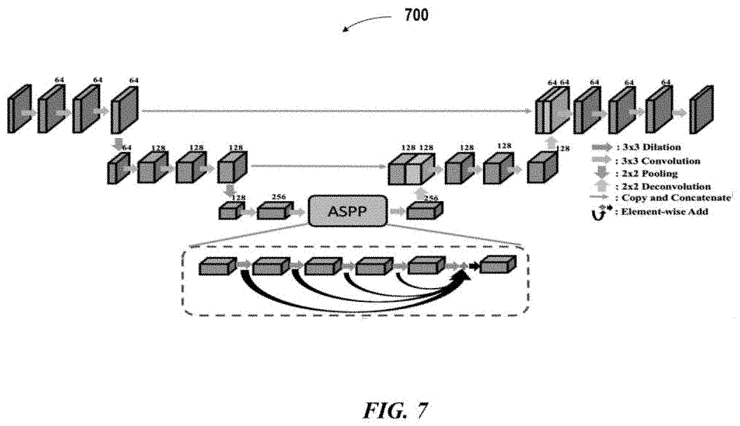

FIG. 7 shows an example of the model architecture 700. As shown in the example, the model architecture may employ Atous Spatial Pyramid Pooling (ASPP) technique. Similar to the training method described above, the two sub-networks may be trained as an integral system using an end-to-end training. Similarly, Dice loss function may be used to determine accurate ROI segmentation result and the weighted sum of Dice loss and boundary loss may be utilized as the total loss. Below is an example of the total loss:

$${ℒ}_{general - DICE} ( ρ , \hat{ρ} ) = 1 - \frac{2 〈 ρ , \hat{ρ} 〉}{{‖ ρ ‖}_{2}^{2} + {‖ \hat{ρ} ‖}_{2}^{2}} \\ {ℒ}_{B} ( θ ) = {\int }_{Ω} {ϕ}_{G} ( q ) {s}_{θ} ( q ) dq \\ {ℒ}_{total} = ( 1 - α ) {ℒ}_{B} + α {ℒ}_{general - DICE}$$

As described above, by training the self-attention subnetwork and the adaptive deep learning subnetwork concurrently in an end-to-end training process, the deep learning subnetwork for enhancing image quality can beneficially adapt to the attention map (e.g., lesion map) to better improve the image quality by leveraging the ROI knowledge.

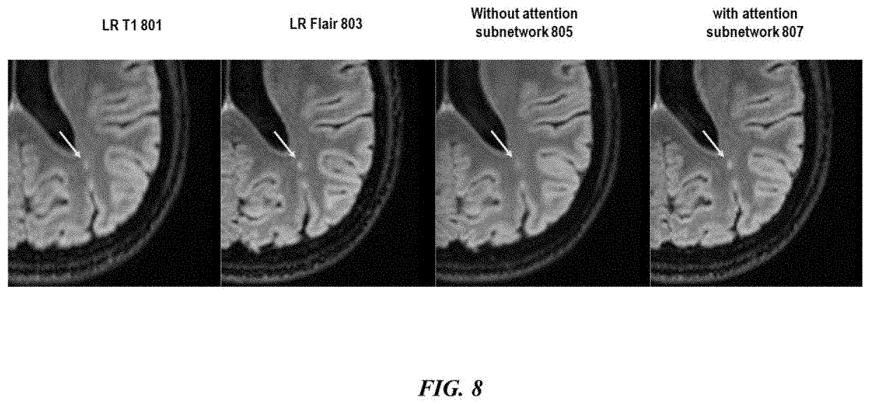

FIG. 8 shows an example of applying the deep learning self-attention mechanism to MR images. As shown in the example, image 805 is an image enhanced over the low-resolution T1 801 and low-resolution FLAIR 803 using a conventional deep learning model without the self-attention subnetwork. Comparing to the image 807 that is generated by the presented model which includes the self-attention subnetwork, image 807 has better image quality showing that deep learning self-attention mechanism and the adaptive deep learning model provide a better image quality.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A computer-implemented method for improving image quality comprising:
(a) acquiring, using a medical imaging apparatus, a medical image of a subject, wherein the medical image is acquired with shortened scanning time or reduced amount of tracer dose; and
(b) applying a deep learning network model to the medical image to generate one or more attention feature maps and an enhanced medical image.

2. The computer-implemented method of claim 1, wherein the deep learning network model comprises a first subnetwork for generating the one or more attention feature maps and a second subnetwork for generating the enhanced medical image.

3. The computer-implemented method of claim 2, wherein an input data to the second subnetwork includes the one or more attention feature maps.

4. The computer-implemented method of claim 2, wherein the first subnetwork and the second subnetwork are deep learning networks.

5. The computer-implemented method of claim 2, wherein the first subnetwork and the second subnetwork are trained in an end-to-end training process.

6. The computer-implemented method of claim 5, wherein the second subnetwork is trained to adapt to the one or more attention feature maps.

7. The computer-implemented method of claim 1, wherein the deep learning network model includes a combination of U-net structure and a residual network.

8. The computer-implemented method of claim 1, wherein the one or more attention feature maps include a noise map or lesion map.

9. The computer-implemented method of claim 1, wherein the medical imaging apparatus is a transforming magnetic resonance (MR) device or a Positron Emission Tomography (PET) device.

10. The computer-implemented method of claim 1, wherein the enhanced medical image has a higher resolution or improved signal-noise ratio.

11. A non-transitory computer-readable storage medium including instructions that, when executed by one or more processors, cause the one or more processors to perform operations comprising:
(a) acquiring, using a medical imaging apparatus, a medical image of a subject, wherein the medical image is acquired with shortened scanning time or reduced amount of tracer dose; and
(b) applying a deep learning network model to the medical image to generate one or more attention feature maps and an enhanced medical image.

12. The non-transitory computer-readable storage medium of claim 11, wherein the deep learning network model comprises a first subnetwork for generating the one or more attention feature maps and a second subnetwork for generating the enhanced medical image.

13. The non-transitory computer-readable storage medium of claim 12, wherein an input data to the second subnetwork includes the one or more attention feature maps.

14. The non-transitory computer-readable storage medium of claim 12, wherein the first subnetwork and the second subnetwork are deep learning networks.

15. The non-transitory computer-readable storage medium of claim 12, wherein the first subnetwork and the second subnetwork are trained in an end-to-end training process.

16. The non-transitory computer-readable storage medium of claim 15, wherein the second subnetwork is trained to adapt to the one or more attention feature maps.

17. The non-transitory computer-readable storage medium of claim 11, wherein the deep learning network model includes a combination of U-net structure and a residual network.

18. The non-transitory computer-readable storage medium of claim 11, wherein the one or more attention feature maps include a noise map or lesion map.

19. The non-transitory computer-readable storage medium of claim 11, wherein the medical imaging apparatus is a transforming magnetic resonance (MR) device or a Positron Emission Tomography (PET) device.

20. The non-transitory computer-readable storage medium of claim 11, wherein the enhanced medical image has a higher resolution or improved signal-noise ratio.
